(19) United States
(12) Patent Application Publication
(10) Pub. No.: US 2021/0301000 A1
(43) Pub. Date: Sep. 30, 2021

(54) NS1-Binding Protein and Uses Thereof

(71) Applicant: GUAGNDONG FEIPENG BIOLOGICAL CO., LTD., Dongguan, Guangdong (CN)

(72) Inventors: Peng CUI, Dongguan, Guangdong (CN); Zhiqiang HE, Dongguan, Guangdong (CN); Yuan MENG, Dongguan, Guangdong (CN); Dongmei ZHONG, Dongguan, Guangdong (CN)

(21) Appl. No.: 17/267,025

(22) PCT Filed: Aug. 26, 2019
(86) PCT No.: PCT/CN2019/102631

§ 371 Date: Feb. 9, 2021

(30) Foreign Application Priority Data
Aug. 28, 2018 (CN) 201810999045.2

(51) Int. Cl. C07K 16/10; G01N 33/569

(52) U.S. Cl. CPC C07K 16/1081; G01N 33/56983; C07K 2317/24; G01N 2333/185; C07K 2317/51; C07K 2317/565; C07K 2317/92; C07K 2317/515

(57) ABSTRACT

Provided is an isolated binding protein including an antigen binding domain binding to an NS1 protein. The binding protein can identify and bind to the NS1 protein so as to detect dengue virus.

### CROSS-REFERENCE TO RELATED APPLICATIONS

The present application is a National Stage of International Patent Application No: PCT/CN2019/102631 filed on Aug. 26, 2019, which claims the benefit of priority to Chinese patent application No. 201810999045.2 filed to the China National Intellectual Property Administration on Aug. 28, 2018 and entitled “NS1-Binding protein and uses thereof”, which is herein incorporated by reference in its entirety.

### SEQUENCE LISTING

The present application contains a Sequence Listing which has been submitted electronically in ASCII format and is hereby incorporated by reference in its entirety. Said ASCII copy is named PN148750_Sequence_listing.txt and is 16.0 kilobytes in size, and contains 16 new sequences from SEQ ID NO:13 to SEQ ID NO:28 described in claims and examples of this file, but not numbered. The original sequences of SEQ ID NO: 1 to SEQ ID NO:12 are identical to the sequence listing filed in the corresponding international application No. PCT/CN2019/102631 filed on Aug. 26, 2019.

### TECHNICAL FIELD

The present disclosure relates to the field of biotechnology and medical technology, in particular to an NS1-binding protein and uses thereof.

### BACKGROUND

Dengue fever (DF), which is an acute mosquito-borne infectious disease caused by four serotypes of viruses (DENV-1, DENV-2, DENV-3, and DENV-4), is primarily spread by Aedes aegypti and Aedes albopictus. DF is an insect-borne viral disease with the widest distribution, the most incidence and great harm, and is widely prevalent in more than 100 countries and regions of Africa, America, Southeast Asia and the Western Pacific of tropical and subtropical regions worldwide.

Clinically, DF is a severe influenza-like disease, mainly manifested by sudden onset of illness, high fever, severe headache, retroorbital pain, muscular and joint pain, with accompanying rash, lymphadenectasis and leukopenia, which can affect all people with symptoms variable depending on the age of the patient. This type of disease is commonly referred to as classical dengue fever, which spreads rapidly and can cause large-scale epidemics. The prevalence rate of dengue fever in susceptible population was 40%-50%, which could reach 80%-90%, but the mortality was very low. Dengue hemorrhagic fever that is characterized with high fever, bleeding, hepatomegaly, severe cases of circulatory failure, and high mortality, is a more serious clinical type. Dengue fever accompanied by shock syndrome is called dengue shock syndrome.

There is no specific treatment for dengue fever. The mortality of dengue hemorrhagic fever will exceed 20% without appropriate treatment, and will be less than 1% after effective supportive therapy. Essential points for the diagnosis of dengue fever are as follows: 1) epidemiological data, activities in the 15 days before the onset, whether you have been to endemic areas, mosquito bites; 2) clinical features, sudden onset, fever, “three pains with three redness” (headache, lumbago, and orbital pain with redness in face, neck, and upper chest), rash; and 3) laboratory examination, white blood cells and platelets decline; detecting positive serum characteristic IgM; IgG in the recovery phase increased by 4-fold over the acute phase; the virus or specific antigen is isolated. The clinical methods used for detection of dengue virus include virus culture, serological detection, virus nucleic acid detection. Virus isolation takes too long time to achieve rapid diagnosis; and conventional serological diagnosis is interfered by extensive cross-reactions,

The specific monoclonal antibody of the NS1 protein in the art has low activity and poor affinity, and cannot be well applied to the detection of the NS1 protein; therefore, there is a strong demand in the art for antibodies that effectively and specifically bind to and detect the NS1 protein.

### SUMMARY

The present disclosure provides an isolated binding protein including an antigen binding domain, where the antigen binding domain includes at least one complementarity determining region selected from the following amino acid sequences, or has at least 80% sequence identity with the complementarity determining region of the following amino acid sequences and has an affinity of K_{D}≤6.55×10⁻⁸ mol/L to an NS1 protein;

the complementarity determining region CDR-VH1 is G-Y-X1-F-T-X2-Y-W-1-G, herein,

X1 is S or T, and X2 is D or E;

the complementarity determining region CDR-VH2 is D-M-X1-P-G-D-X2-Y-1-N-Y-X3-E-K-F-K-G, herein,

X1 is F or V, X2 is V, L or I, and X3 is Q or N;

the complementarity determining region CDR-VH3 is T-N-F-X1-T-X2-G-G-X3-D-Y, herein,

X1 is I or L, X2 is L or V, and X3 is V, L or I;

the complementarity determining region CDR-VL1 is K-S-S-X1-S-L-L-X2-S-D-G-X3-T-Y-L-N, herein,

X1 is Q or N, X2 is E or D, and X3 is R or K;

the complementarity determining region CDR-VL2 is L-V-X1-K-X2-D-S, herein,

X1 is S or T, and X2 is V, I or L;

the complementarity determining region CDR-VL3 is W-X1-G-T-H-F-X2-H-T, herein,

X1 is Q, Y or W, and X2 is A or P.

In one or more embodiments, in the complementarity determining region CDR-VH1, X1 is T;

in the complementarity determining region CDR-VH2, X1 is F;

in the complementarity determining region CDR-VH3, X2 is L;

in the complementarity determining region CDR-VL1, X2 is D;

in the complementarity determining region CDR-VL2, X1 is S; and in the complementarity determining region CDR-VL3, X2 is P.

In one or more embodiments, in the complementarity determining region CDR-VH1, X2 is D;

in one or more embodiments, in the complementarity determining region CDR-VH1, X2 is E;

in one or more embodiments, in the complementarity determining region CDR-VH2, X2 is V, and X3 is Q;

in one or more embodiments, in the complementarity determining region CDR-VH2, X2 is V, and X3 is N;

in one or more embodiments, in the complementarity determining region CDR-VH2, X2 is L, and X3 is Q;

in one or more embodiments, in the complementarity determining region CDR-VH2, X2 is L, and X3 is N;

in one or more embodiments, in the complementarity determining region CDR-VH2, X2 is I, and X3 is Q;

in one or more embodiments, in the complementarity determining region CDR-VH2, X2 is I, and X3 is N;

in one or more embodiments, in the complementarity determining region CDR-VH3, X1 is I, and X3 is V;

in one or more embodiments, in the complementarity determining region CDR-VH3, X1 is I, and X3 is L;

in one or more embodiments, in the complementarity determining region CDR-VH3, X1 is I, and X3 is I;

in one or more embodiments, in the complementarity determining region CDR-VH3, X1 is L, and X3 is V;

in one or more embodiments, in the complementarity determining region CDR-VH3, X1 is L, and X3 is L;

in one or more embodiments, in the complementarity determining region CDR-VH3, X1 is L, and X3 is I;

in one or more embodiments, in the complementarity determining region CDR-VL1, X1 is Q, and X2 is R;

in one or more embodiments, in the complementarity determining region CDR-VL1, X1 is Q, and X2 is K;

in one or more embodiments, in the complementarity determining region CDR-VL1, X1 is N, and X2 is R;

in one or more embodiments, in the complementarity determining region CDR-VL1, X1 is N, and X2 is K;

in one or more embodiments, in the complementarity determining region CDR-VL2, X2 is V;

in one or more embodiments, in the complementarity determining region CDR-VL2, X2 is I;

in one or more embodiments, in the complementarity determining region CDR-VL2, X2 is L;

in one or more embodiments, in the complementarity determining region CDR-VL3, X1 is Q;

in one or more embodiments, in the complementarity determining region CDR-VL3, X1 is Y;

in one or more embodiments, in the complementarity determining region CDR-VL3, X1 is W.

In one or more embodiments, the amino acids at the corresponding sites of the complementarity determining regions are listed below,

| | CDR- | CDR- | CDR- | CDR- | CDR- | CDR- |
|---|---|---|---|---|---|---|
| | VH1 | VH2 | VH3 | VL1 | VL2 | VL3 |
| Sites | X2 | X2/X3 | X1/X3 | X1/X3 | X2 | X1 |
| Mutation 1 | D | L/Q | I/L | Q/R | V | Q |
| Mutation 1-1 | E | L/N | I/L | Q/K | I | Y |
| Mutation 1-2 | D | V/Q | I/V | N/R | L | W |
| Mutation 1-3 | E | V/N | L/I | N/K | V | Q |
| Mutation 1-4 | D | I/O | L/L | Q/R | I | Y |
| Mutation 1-5 | E | I/N | L/V | Q/K | L | W |
| Mutation 1-6 | D | L/N | I/I | Q/R | V | Q |
| Mutation 1-7 | E | L/Q | I/L | Q/K | I | Y |
| Mutation 1-8 | D | L/N | I/V | N/R | L | W |
| Mutation 1-9 | E | V/Q | L/I | N/K | V | Q |
| Mutation 1-10 | D | V/N | L/L | Q/R | I | Y |
| Mutation 1-11 | E | I/Q | L/V | Q/K | L | W |
| Mutation 1-12 | D | I/N | I/I | Q/R | V | Q |
| Mutation 1-13 | E | V/Q | I/V | Q/R | I | Y |
| Mutation 1-14 | D | V/N | I/I | Q/K | L | Q |
| Mutation 1-15 | E | I/Q | I/I | N/R | V | Q |
| Mutation 1-16 | D | I/N | L/L | N/K | I | Y |
| Mutation 1-17 | E | V/N | I/L | Q/R | L | W |
| Mutation 1-18 | D | I/Q | I/V | Q/K | V | Q |
| Mutation 1-19 | E | I/N | I/I | N/R | I | Y |
| Mutation 1-20 | D | I/Q | I/L | Q/R | L | W |
| Mutation 1-21 | E | I/N | I/V | Q/K | V | Q |
| Mutation 1-22 | D | I/N | I/L | Q/R | I | Y |
| Mutation 1-23 | E | L/Q | I/V | Q/R | L | W |
| Mutation 1-24 | D | L/N | I/I | Q/K | V | Q |
| Mutation 1-25 | E | V/Q | L/L | N/R | I | Y |
| Mutation 1-26 | D | V/N | L/V | N/K | L | W |
| Mutation 1-27 | E | I/Q | L/I | Q/K | V | Q |
| Mutation 1-28 | D | I/N | I/I | N/R | I | Y |
| Mutation 1-29 | E | L/Q | L/L | N/K | L | W |
| Mutation 1-30 | E | L/N | L/V | N/K | I | W |
| Mutation 1-31 | E | V/Q | L/I | Q/K | I | Y |
| Mutation 1-32 | D | V/N | L/L | N/R | L | W |
| Mutation 1-33 | E | I/Q | L/V | N/K | V | Q |
| Mutation 1-34 | D | I/N | L/I | Q/R | I | Y |
| Mutation 1-35 | E | L/Q | L/V | Q/K | L | W |
| Mutation 1-36 | D | L/N | L/I | N/R | V | Q |
| Mutation 1-37 | E | V/Q | L/I | N/K | I | Y |
| Mutation 1-38 | D | V/N | I/L | Q/R | L | W |
| Mutation 1-39 | E | I/Q | I/V | Q/K | V | Q |
| Mutation 1-40 | D | V/Q | I/I | N/R | I | Y |
| Mutation 1-41 | E | L/Q | L/L | N/K | L | Y |
| Mutation 1-42 | D | L/N | L/V | Q/R | V | Q |
| Mutation 1-43 | E | V/Q | L/I | Q/K | I | Q |
| Mutation 1-44 | D | V/N | I/L | N/R | L | W |
| Mutation 1-45 | E | I/Q | I/V | N/K | V | Q |
| Mutation 1-46 | E | I/N | I/I | N/R | V | Y |
| Mutation 1-47 | E | L/Q | L/L | N/K | L | W |
| Mutation 1-48 | D | L/N | L/V | N/R | V | Q |
| Mutation 1-49 | E | V/Q | L/I | N/K | I | W |
| Mutation 1-50 | D | V/N | I/V | N/K | L | W |
| Mutation 1-51 | D | L/Q | I/I | Q/R | V | Q |
| Mutation 1-52 | E | V/Q | L/V | Q/R | L | W |

In one or more embodiments, the binding protein includes at least 3 CDRs; or the binding protein includes at least 6 CDRs.

In one or more embodiments, the binding protein is one of a nanobody, F(ab′)2, Fab′, Fab, Fv, scFv, a bispecific antibody, and a minimum recognition unit of an antibody.

In one or more embodiments, the binding protein includes the light chain framework regions FR-L1, FR-L2, FR-L3, and FR-L4 with sequences shown in SEQ ID NOs: 1-4 successively, and/or the heavy chain framework regions FR-H1, FR-H2, FR-H3, and FR-H4 with sequences shown in SEQ ID NOs: 5-8 successively.

In one or more embodiments, the binding protein further includes an antibody constant region sequence;

for example, the constant region sequence is selected from the sequence of any one of the constant regions of IgG1, IgG2, IgG3, IgG4, IgA, IgM, IgE, and IgD.

For example, the species source of the constant region is cattle, horse, dairy cow, pig, sheep, goat, rat, mouse, dog, cat, rabbit, camel, donkey, deer, mink, chicken, duck, goose, turkey, gamecock, or human.

For example, the constant region is derived from a mouse;

a light chain constant region sequence is shown in SEQ ID NO: 9; and

a heavy chain constant region sequence is shown in SEQ ID NO: 10.

The present disclosure also provides an isolated nucleic acid encoding the binding protein.

The present disclosure also provides a vector including the nucleic acid.

The present disclosure also provides a host cell including the nucleic acid or the above-described vector.

The present disclosure also provides a method for producing the binding protein, which includes the following steps: culturing the host cell in a culture medium, and recovering the produced binding protein from the culture medium or from the cultured host cell.

The present disclosure also provides a use of the binding protein in preparing a product for detecting dengue infection.

The present disclosure also provides a kit including one or more of the binding protein, the isolated nucleic acid, or the vector.

The present disclosure also provides a use of the binding protein in detecting dengue infection.

The present disclosure also provides a method for detecting dengue infection, which includes the following steps: A) contacting a sample from a subject with the binding protein for performing a binding reaction in a condition sufficient for a binding reaction; and B) detecting an immune complex generated by the binding reaction; herein the presence of the immune complex is indicative of the presence of the dengue infection.

The present disclosure provides an isolated binding protein including an antigen binding domain binding to an NS1 protein, the binding protein with higher sensitivity and specificity can specifically identify and bind to the NS1 protein, thereby enabling detection of dengue virus. Moreover, there is no need to use mouse peritoneal cavity to induce hybridoma cells to produce the binding protein, which has the advantages of being less difficult in the production, and more stable in the antibody function.

### BRIEF DESCRIPTION OF DRAWINGS

To describe the technical solutions in the specific embodiments of the present disclosure or the prior art more clearly, the following briefly introduces accompanying drawings required for describing the specific embodiments or the prior art. Apparently, the accompanying drawings in the following description show one or more of embodiments of the present disclosure, and a person of ordinary skill in the art may still derive other accompanying drawings from these accompanying drawings without creative efforts.

FIG. 1 is an electropherogram of monoclonal antibodies against the NS1 protein provided in Example 1 of the present disclosure.

### DETAILED DESCRIPTION

The present disclosure can be more easily understood by reference to the subsequent description of some embodiments of the present disclosure and the detailed content of the Examples included therein.

Before the present disclosure is described further, it is to be understood that this present disclosure is not limited to the specific embodiments described, since these embodiments are necessarily diverse. It should also be understood that the terms used in this specification are only intended to illustrate specific embodiments, rather than as limitations, because the scope of the present disclosure may only be defined in the appended claims.

Unless otherwise defined herein, scientific and technical terms used in connection with this disclosure shall have the meanings commonly understood by those of ordinary skill in the art. The meaning and scope of the terms should be clear, however, given any potential ambiguity, the definitions provided herein take precedence over any dictionary or foreign definitions. In the present application, the use of “or” means “and/or” unless otherwise indicated. In addition, the use of the term “including” and other forms is non-limiting.

In general, a nomenclature and technology thereof used in connection with cell and tissue culture, molecular biology, immunology, microbiology, genetics, and protein and nucleic acid chemistry and hybridization described herein are those well-known and commonly used in the art. Unless otherwise indicated, the methods and technology of the present disclosure are generally performed according to conventional methods well known in the art, and as described in various general and more specific references, which are cited and discussed throughout this specification. Enzymatic reactions and purification technology are performed according to the manufacturer's instructions, as commonly achieved in the art or as described herein. The nomenclature used in conjunction with the analytical chemistry, synthetic organic chemistry, and medical and pharmaceutical chemistry described herein, as well as the laboratory procedures and technology thereof, are those well known and commonly used in the art.

Selected terms are defined below for more easily understanding of the present disclosure.

The term “amino acid” refers to a naturally existing or non-naturally existing carboxyl α-amino acid. The term “amino acid” as used herein may include both naturally existing amino acids and non-naturally existing amino acids. Naturally existing amino acids include alanine (three-letter code: Ala, single-letter code: A), arginine (Arg, R), asparagine (Asn, N), aspartic acid (Asp, D), cysteine (Cys, c), glutamine (Gln, Q), glutamic acid (Glu, E), glycine (Gly, G), histidine (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V). Non-naturally existing amino acids include, but are not limited to, α-aminoadipic acid, aminobutyric acid, citrulline, homocysteine, homoleucine, homoarginine, hydroxyproline, norleucine, pyridylalanine, and sarcosine.

The term “isolated binding protein” is a protein that does not bind to a naturally-bound component which is associated with it in its natural state due to its derived origin or source, substantially free of other proteins from the same species; expressed by cells from different species; or does not exist in nature. Therefore, a protein that is chemically synthesized or synthesized in a cell system different from the cell of its natural origin will be “isolated” from its naturally-bound component. Proteins can also be made substantially free of naturally-bound components by isolation, for example, using protein purification technology well known in the art.

The term “isolated binding protein including an antigen binding domain” generally refers to all proteins/protein fragments including CDR regions. As used herein, the term “antibody” includes polyclonal and monoclonal antibodies and antigen compound binding fragments of these antibodies, including Fab, F(ab′)2, Fd, Fv, scFv, bispecific antibodies, and a minimum recognition unit of an antibody, as well as single chain derivatives of these antibodies and fragments. The type of antibodies may be selected from IgG1, IgG2, IgG3, IgG4, IgA, IgM, IgE, and IgD. Furthermore, the term “antibody” includes naturally existing antibodies as well as non-naturally existing antibodies, including, for example, chimeric, bifunctional and humanized antibodies, and related synthetic isoforms. As used herein, the term “antibody” is used interchangeably with “immunoglobulin”.

“Variable region” or “variable domain” of an antibody refers to the amino-terminal domain of the heavy or light chain of the antibody. The variable domain of the heavy chain may be referred to as “VH”. The variable domain of the light chain may be referred to as “VL”. These domains are typically the most variable portions of an antibody and contain antigen binding sites. The variable region of the light chain or heavy chain is composed of three hypervariable regions called “complementarity determining regions” or “CDRs” and a framework region (FR) separating them. The framework region of the antibody plays a role in positioning and aligning CDRs; the CDRs are mainly responsible for binding to the antigen.

As used herein, “framework region” or “FR” means a region of an antibody variable domain excluding those regions defined as CDR. Each antibody variable domain framework region may be further subdivided into adjacent regions (FR1, FR2, FR3 and FR4) separated by CDRs. As used herein, the term “bispecific antibody” or “bifunctional antibody” refers to an artificial hybrid binding protein having two different pairs of heavy/light chains and two different binding sites. Bispecific binding proteins can be produced by a variety of methods, including fusion of hybridomas or ligation of Fab′ fragments.

As used herein, the term “sequence identity” refers to the similarity between at least two different sequences. This percentage identity can be determined by standard algorithms, such as Basic Local Alignment Search Tool (BLAST); Needleman-Wunsch Algorithm; or Myers' Diff Algorithm. In one or more embodiments a set of parameters may be the Blosum 62 scoring matrix and gap penalty of 12, gap extension penalty of 4, and frame shift gap penalty of 5. In one or more embodiments the percentage identity between two amino acid or nucleotide sequences can also be determined using the Myers-Miller algorithm ((1989) CABIOS 4: 11-17), which has been incorporated into the ALIGN program (version 2.0), using a PAM120 weight residue table, a gap length penalty of 12, and a gap penalty of 4. Percentage identity is typically calculated by comparing sequences with a similar length.

In general, the variable region VL/VH of heavy chain and light chain can be obtained by the following combination of CDRs and FRs with the following number: FR1-CDR1-FR2-CDR2-FR3-CDR3-FR4.

As used herein, the term “purified” or “isolated” in relation to a polypeptide or a nucleic acid means that the polypeptide or the nucleic acid is not in its natural medium or in its natural form. Thus, the term “isolated” includes removal of a polypeptide or a nucleic acid from its original environment, for example if it is naturally existing, from the natural environment. For example, an isolated polypeptide usually does not contain at least some protein or other cellular components normally associated therewith or normally mixed therewith or in a solution. An isolated polypeptide includes the naturally produced polypeptide contained in a cell lysate, the polypeptide in purified or partially purified form, a recombinant polypeptide, the polypeptide expressed or secreted by a cell, and the polypeptide in a heterologous host cell or culture. In connection with a nucleic acid, the term isolated or purified indicates, for example, that the nucleic acid is not in its natural genomic background (e.g., in a vector, as an expression cassette, linked to a promoter, or artificially introduced into a heterologous host cell).

The present disclosure provides an isolated binding protein including an antigen binding domain, where the antigen binding domain includes at least one complementarity determining region selected from the following amino acid sequences, or has at least 80% sequence identity with the complementarity determining region of the following amino acid sequence and has an affinity of K_{D}≤6.55×10⁻⁸ mol/L to an NS1 protein;

the complementarity determining region CDR-VH1 is G-Y-X1-F-T-X2-Y-W-1-G (SEQ ID NO:13), herein,

X1 is S or T, and X2 is D or E;

the complementarity determining region CDR-VH2 is D-M-X1-P-G-D-X2-Y-1-N-Y-X3-E-K-F-K-G (SEQ ID NO:14), herein,

X1 is F or V, X2 is V, L or I, and X3 is Q or N;

the complementarity determining region CDR-VH3 is T-N-F-X1-T-X2-G-G-X3-D-Y (SEQ ID NO:15), herein,

X1 is I or L, X2 is L or V, and X3 is V, L or I;

the complementarity determining region CDR-VL1 is K-S-S-X1-S-L-L-X2-S-D-G-X3-T-Y-L-N(SEQ ID NO:16), herein,

X1 is Q or N, X2 is E or D, and X3 is R or K;

the complementarity determining region CDR-VL2 is L-V-X1-K-X2-D-S(SEQ ID NO:17), herein,

X1 is S or T, and X2 is V, I or L;

the complementarity determining region CDR-VL3 is W-X1-G-T-H-F-X2-H-T (SEQ ID NO:18), herein,

X1 is Q, Y or W, and X2 is A or P.

In one or more embodiments, X1 appearing in the six CDR regions of the binding protein described in the present disclosure each independently represents an amino acid defined in the present disclosure; X2 appearing in the six CDR regions of the binding protein described in the present disclosure each independently represents an amino acid defined in the present disclosure; and X3 appearing in the six CDR regions of the binding protein described in the present disclosure each independently represents an amino acid defined in the present disclosure.

It is well known in the art that the binding specificity and affinity of an antibody is determined mainly by CDR sequences, and that variants with similar biological activity can be readily obtained by modifying the amino acid sequence of non-CDR regions according to well-established, well-known technology. Thus, the present disclosure also includes “functional derivatives” of the binding proteins. The “functional derivative” refers to a variant of an amino acid substitution, and a functional derivative retains detectable binding protein activity, e.g., the activity of an antibody capable of binding to the NS1 protein. The “functional derivatives” may include “variants” and “fragments” that have similar biological activities because they have exactly the same CDR sequences as the binding proteins described in the present disclosure.

In one or more embodiments, the antigen binding domain has at least 50%, or at least 55%, or at least 60%, or at least 65%, or at least 70%, or at least 75%, or at least 80%, or at least 85%, or at least 90%, or at least 91%, or at least 92%, or at least 93%, or at least 94%, or at least 95%, or at least 96%, or at least 97%, or at least 98%, or at least 99% sequence identity with the complementarity determining region of the following amino acid sequences and has an affinity of KD≤6.550×10⁻⁸ mol/L with the NS1 protein, for example, the KD value may be 4.587×10⁻⁸ mol/L, 2.798×10⁻⁸ mol/L, 1.282×10⁻⁸ mol/L, 1.891×10⁻⁹ mol/L, 4.601×10−9 mol/L, 6.592×10⁻⁹ mol/L, 8.002×10⁻⁹ mol/L, 9.614×10⁻⁹ mol/L, 2.324×10⁻¹⁰ mol/L, 3.540×10⁻¹⁰ mol/L, 6.121×10⁻¹⁰ mol/L, 9.876×10⁻¹⁰ mol/L, or 2.324×10⁻¹⁰ mol/L≤KD≤6.550×10⁻⁸ mol/L, or 2.324×10⁻¹⁰ mol/L≤KD≤9.614×10⁻⁹ mol/L, or KD≤1.03×10⁻¹⁰ mol/L, ≤1.15×10⁻¹⁰ mol/L, ≤1.21×10⁻¹⁰ mol/L, ≤0.32×10⁻⁹ mol/L, ≤0.46×10⁻⁹ mol/L, ≤0.70×10⁻⁹ mol/L, ≤0.80×10⁻⁹ mol/L, ≤0.90×10⁻⁹ mol/L, ≤1.07×10⁻⁹ mol/L, or ≤1.14×10⁻⁹ mol/L. Herein, the affinity is determined according to the method in the description of the present disclosure.

In one or more embodiments,

in the complementarity determining region CDR-VH1, X1 is T; in the complementarity determining region CDR-VH2, X1 is F; in the complementarity determining region CDR-VH3, X2 is L; in the complementarity determining region CDR-VL1, X2 is D; in the complementarity determining region CDR-VL2, X1 is S; and in the complementarity determining region CDR-VL3, X2 is P.

In one or more embodiments, in the complementarity determining region CDR-VH1, X2 is D.

In one or more embodiments, in the complementarity determining region CDR-VH1, X2 is E.

In one or more embodiments, in the complementarity determining region CDR-VH2, X2 is V, and X3 is Q.

In one or more embodiments, in the complementarity determining region CDR-VH2, X2 is V, and X3 is N.

In one or more embodiments, in the complementarity determining region CDR-VH2 X2 is L, and X3 is Q.

In one or more embodiments, in the complementarity determining region CDR-VH2, X2 is Land X3 is N.

In one or more embodiments, in the complementarity determining region CDR-VH2 X2 is I, and X3 is Q.

In one or more embodiments, in the complementarity determining region CDR-VH2, X2 is I, and X3 is N.

In one or more embodiments, in the complementarity determining region CDR-VH3, X1 is I, and X3 is V.

In one or more embodiments, in the complementarity determining region CDR-VH3, X1 is I, and X3 is L.

In one or more embodiments, in the complementarity determining region CDR-VH3, X1 is I, and X3 is I.

In one or more embodiments, in the complementarity determining region CDR-VH3, X1 is L, and X3 is V.

In one or more embodiments, in the complementarity determining region CDR-VH3, X1 is L, and X3 is L.

In one or more embodiments, in the complementarity determining region CDR-VH3, X1 is L, and X3 is I.

In one or more embodiments, in the complementarity determining region CDR-VL1, X1 is Q, and X2 is R.

In one or more embodiments, in the complementarity determining region CDR-VL1, X1 is Q, and X2 is K.

In one or more embodiments, in the complementarity determining region CDR-VL1, X1 is N, and X2 is R.

In one or more embodiments, in the complementarity determining region CDR-VL1, X1 is N, and X2 is K.

In one or more embodiments, in the complementarity determining region CDR-VL2, X2 is V.

In one or more embodiments, in the complementarity determining region CDR-VL2, X2 is I.

In one or more embodiments, in the complementarity determining region CDR-VL2, X2 is L.

In one or more embodiments, in the complementarity determining region CDR-VL3, X1 is Q.

In one or more embodiments, in the complementarity determining region CDR-VL3, X1 is Y.

In one or more embodiments, in the complementarity determining region CDR-VL3, X1 is W.

In one or more embodiments, the binding protein includes at least 3 CDRs; or, the binding protein includes at least 6 CDRs.

In one or more embodiments, the binding protein is a complete antibody including a variable region and a constant region.

In one or more embodiments, the binding protein is one of a nanobody, F(ab′)2, Fab′, Fab, Fv, scFv, a bispecific antibody, and a minimum recognition unit of an antibody.

In one or more embodiments, the binding protein includes the light chain framework regions FR-L1, FR-L2, FR-L3, and FR-L4 with sequences shown in SEQ ID NOs: 1-4 successively, and/or the heavy chain framework regions FR-H1, FR-H2, FR-H3, and FR-H4 with sequences shown in SEQ ID NOs: 5-8 successively.

In one or more embodiments, the binding protein further includes an antibody constant region sequence; for example, the constant region sequence is selected from the sequence of any one of the constant regions of IgG1, IgG2, IgG3, IgG4, IgA, IgM, IgE, and IgD.

In one or more embodiments, the species of the constant region is derived from cattle, horse, dairy cow, pig, sheep, goat, rat, mouse, dog, cat, rabbit, camel, donkey, deer, mink, chicken, duck, goose, turkey, gamecock, or human; for example, the constant region is derived from mouse;

a light chain constant region sequence is shown in SEQ ID NO: 9; and

a heavy chain constant region sequence is shown in SEQ ID NO: 10.

The present disclosure also provides an isolated nucleic acid encoding the above-described binding protein.

As used herein, nucleic acids include conservatively substituted variants thereof (for example, degenerate codons substitution) and complementary sequences. The terms “nucleic acid” and “polynucleotide” are synonymous and include genes, cDNA molecules, mRNA molecules, and fragments thereof such as oligonucleotides.

The present disclosure also provides a vector including the nucleic acid. Herein the nucleic acid sequence is operably linked to at least one regulatory sequence. The “operably linked” is intended to mean that the coding sequence is linked to a regulatory sequence in a manner which allows for the expression of the coding sequence. The regulatory sequences are selected to direct expression of the target protein in a suitable host cell, including promoters, enhancers and other expression regulatory elements.

As used herein, a vector may refer to a molecule or agent including a nucleic acid or a fragment thereof of the present disclosure, carrying genetic information and delivering the genetic information to a cell. Typical vectors include plasmids, viruses, phages, cosmids and minichromosomes. The vector may be a cloning vector (i.e., a vector for transferring the genetic information into cells, the cells may be propagated and the cells with or without genetic information may be screened), or an expression vector (i.e., a vector including the necessary genetic elements to permit expression of the genetic information of the vector in a cell). Thus, the cloning vector may contain a selectable marker and an origin of replication that matches the cell type specified by the cloning vector, while the expression vector contains regulatory elements necessary to express in the specified target cell.

The nucleic acids or fragments thereof of the present disclosure can be inserted into suitable vectors to form cloning vectors or expression vectors carrying the nucleic acid fragments of the present disclosure. Such novel vectors are also part of the present disclosure. The vectors may include plasmids, bacteriophages, cosmids, minichromosomes or viruses, as well as naked DNA that is only transiently expressed in a particular cell. The cloning vectors and expression vectors of the present disclosure are capable of autonomous replication, and thus are capable of providing high copy numbers for the purposes of high-level expression or high-level replication for subsequent cloning. The expression vector may include a promoter for driving expression of a nucleic acid fragment of the present disclosure, optionally a nucleic acid sequence encoding a signal peptide that allows the peptide expression product to be secreted or integrated onto a membrane, a nucleic acid fragment of the present disclosure, and optionally a nucleic acid sequence encoding a terminator. When the expression vector is manipulated in a production strain or a cell line, the vector may or may not be integrated into the genome of the host cell when introduced into the host cell. Vectors usually carry replication sites, as well as marker sequences capable of providing phenotypic selection in transformed cells.

The expression vectors of the present disclosure are used for transforming host cells. Such transformed cells are also part of the present disclosure and may be cultured cells or cell lines for propagation of nucleic acid fragments and vectors of the present disclosure, or for recombinant production of polypeptides of the present disclosure. Transformed cells of the present disclosure include microorganisms such as bacteria (e.g., E. coli, Bacillus subtilis etc.). Host cells also include cells from multicellular organisms such as fungi, insect cells, plant cells or mammalian cells, preferably from mammals, e.g., Chinese hamster ovary cells (CHO cells). The transformed cells are capable of replicating the nucleic acid fragments of the present disclosure. When the recombinant production of polypeptides of the present disclosure, the expression products may be exported to the culture medium or carried on the surface of the transformed cells.

The present disclosure also provides a method for producing the binding protein, which includes the following steps:

culturing the host cells in a culture medium, and recovering the produced binding protein from the culture medium or from the cultured host cells.

The method may include the steps of, for example, transfecting a host cell with a nucleic acid vector encoding at least a portion of a binding protein, and culturing the host cell under suitable conditions to express the binding protein. The host cell may also be transfected with one or more expression vectors, which may contain DNA encoding at least a part of the binding protein alone or in combination. The binding proteins can be separated from the culture medium or the cell lysate using conventional technology for purifying proteins and peptides, including ammonium sulfate precipitation, chromatography (e.g., ion exchange, gel filtration, affinity chromatography, etc.), and/or electrophoresis.

The construction of a suitable vector containing the coding and regulatory sequences of interest can be carried out using standard ligation and restriction technology known in the art. The isolated plasmid, DNA sequence or synthetic oligonucleotide is cleaved, tailed and religated as a needed form. Mutations may be introduced into the coding sequence by any method to produce variants of the present disclosure, and such mutations may include deletions or insertions or substitutions, etc.

The present disclosure also provides antibodies that can react with an epitope of the NS1 protein, including monoclonal and polyclonal antibodies. The antibody may include the complete binding protein, or a fragment or a derivative thereof. The preferred antibody includes all or part of the binding protein.

The present disclosure also provides a use of the binding protein in preparing a product for detecting dengue infection.

In one or more embodiments, the binding protein provided by the present disclosure can be used to detect the presence of one or more target molecules in a biological sample, The term “detecting” as used herein includes quantitative or qualitative detection. In one or more embodiments, the biological sample includes a cell or a tissue.

Immunoassays of the present disclosure include colloidal gold immunoassays, as well as ELISA and other assays or methods that utilize antigen-antibody reactions.

As used herein, the term “colloidal gold immunoassay” is an immunolabeling technology using colloidal gold as a tracer marker applied to antigens and/or antibodies. The colloidal gold is formed by polymerizing chloroauric acid into gold particles with a specific size under the action of a reducing agent, such as white phosphorus, ascorbic acid, sodium citrate, and tannic acid, and is in a stable colloidal state due to the electrostatic interaction.

In one or more embodiments, the present disclosure provides an article (e.g., a kit) including materials used for diagnosis of dengue virus infection. The article includes a container and a label or a package instruction on or together with the container. Suitable containers include, for example, bottles or syringes. The container may be made of various materials such as glass or plastic. The container holds a composition, alone or in combination with another composition effective for diagnosing dengue. At least one active agent in the composition is the binding protein provided by the present disclosure.

In one or more embodiments, the present disclosure also provides detection kits including the binding protein, the nucleic acid, or the vector described above.

A method for detecting a NS1 protein antigen in a test sample, including:

A) contacting the NS1 protein antigen in the test sample with the binding protein under a condition sufficient for an antibody/antigen binding reaction to form an immune complex; and

B) detecting the presence of the immune complex, the presence of the complex being indicative of the presence of the NS1 protein antigen in the test sample.

In one or more embodiments, the binding protein may be labeled with an indicator that indicates the intensity of the signal, so that the complex is easily detected.

In one or more embodiments, in step a), the immune complex further includes a second antibody binding to the binding protein; In this embodiment, the binding protein forms a paired antibody with the second antibody in the form of a first antibody for binding to different epitopes of the NS1 protein; The second antibody may be labeled with an indicator that indicates the intensity of the signal, so that the complex is easily detected.

In one or more embodiments, in step a), the immune complex further includes a second antibody binding to the NS1 protein antigen; In this embodiment, the binding protein serves as an antigen for the second antibody, which may be labeled with an indicator that indicates the intensity of the signal, so that the complex is easily detected.

In one or more embodiments, the indicator that indicates the intensity of the signal includes any one of a fluorescent substance, a quantum dot, a digoxin-labeled probe, biotin, a radioisotope, a radioactive contrast agent, a paramagnetic ion fluorescent microsphere, an electron dense substance, a chemiluminescent label, an ultrasound contrast agent, a photosensitizer, colloidal gold, or an enzyme.

In one or more embodiments, the fluorescent material includes any one of Alexa 350, Alexa 405, Alexa 430, Alexa 488, Alexa 555, Alexa 647, AMCA, am inoacridine, BODIPY 630/650, BODIPY 650/665, BODIPY-FL, BODIPY-R6G, BOD IPY-TMR, BODIPY-TRX, 5-carboxy-4′,5′-dichloro-2′,7′-dimethoxyfluorescein, 5-carb oxy-2′,4′,5′,7′-tetrachlorofluorescein, 5-carboxyfluorescein, 5-carboxyrhodamine, 6-carboxyrhodamine, 6-carboxytetramethylrhodamine, Cascade Blue, Cy2, Cy3, Cy5, Cy7, 6-FAM, dansyl chloride, fluorescein, HEX, 6-JOE, NBD (7-nitrobenzo-2-oxa-1,3-diazole), Oregon Green 488, Oregon Green 500, Oregon Green 514, Pacif ic Blue, phthalic acid, terephthalic acid, isophthalic acid, cresyl fast violet, cresyl blue violet, brilliant cresyl blue, p-aminobenzoic acid, erythrosine, phthalocyanin e, azomethine, cyanine, xanthine, succinylfluorescein, rare earth metal cryptates, tris-bispyridyldiamine europium, europium cryptates or chelates, diamines, dicya nin, La Jolla blue dye, allophycocyanin, allococyanin B, phycocyanin C, phycocy anin R, thiamine, phycoerythrin/phycocyanin, phycoerythrin R, REG, rhodamine g reen, rhodamine isothiocyanate, rhodamine red, ROX, TAMRA, TET, TRIT (Tetra methylRhodamine Isothiol), tetramethyl rhodamine and Texas red.

In one or more embodiments, the radioisotope includes any one of ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ⁹⁴mTc, ⁹⁴Tc, ⁹⁹mTc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ⁵²mMn, ⁵⁵Co, ⁷²As, ⁷⁵Br, ⁷⁶Br, ⁸²mRb, and ⁸³Sr.

In one or more embodiments, the enzyme includes any one of horseradish peroxidase, alkaline phosphatase, and glucose oxidase.

In one or more embodiments, the fluorescent microsphere is a polystyrene fluorescent microsphere, and the rare earth fluorescent ions europium is wrapped inside.

In one or more embodiments, the present disclosure provides a kit for determining the presence of NS1 protein in a subject infected with, for example, dengue, the kit comprising at least one binding protein provided by the present disclosure, associated buffers, reagents required for reacting a liquid sample with the binding protein, and reagents for determining the presence of a positive or a negative binding reaction between NS1 protein and the binding protein. In order to determine the presence of the NS1 protein, the kit may, for example, use a binding protein with a label as an antibody, herein the label may be any suitable label, such as a colloidal gold label.

The present disclosure also provides use of the binding protein as described herein in detecting dengue infection.

The present disclosure also provides a method for detecting of dengue infection, which includes the following steps:

A) contacting a sample from a subject with the binding protein as described herein for performing a binding reaction in a condition sufficient for a binding reaction; and

B) detecting an immune complex generated by the binding reaction, herein the presence of the immune complex is indicative of the presence of a dengue infection.

In one or more embodiments, the method is based on a fluorescent antibody technology, a radioimmunoassay, and/or an enzyme immunoassay.

In one or more embodiments, the method is based on an enzyme-linked immunoassay.

In one or more embodiments, the method is based on a colloidal gold immunoassay.

In one or more embodiments, the sample is selected from at least one of whole blood, peripheral blood, serum, or plasma.

In one or more embodiments, the subject is a mammal, for example, a primate, such as a human.

Some examples are provided below to illustrate the disclosure, but not to limit the scope of the disclosure.

### Example 1

The restriction enzyme and Prime Star DNA polymerase, in this example were purchased from Takara. MagExtractor-RNA extraction kit was purchased from TOYOBO. SMARTER RACE cDNA Amplification Kit was purchased from Takara. The pMD-18T vector was purchased from Takara. Plasmid extraction kits were purchased from Tiangen Biotech (Beijing) Co., Ltd. Primer synthesis and gene sequencing were performed by Invitrogen Corporation. The hybridoma cell strain secreting the 11E2 monoclonal antibody against dengue virus NS1 was a hybridoma cell strain newly screened by the laboratory of the present disclosure.

(1) Primers

5′ RACE primers for amplification of heavy chain and light chain:

SMARTER II A oligonucleotide:
(SEQ ID NO: 19)
5′-AAGCAGTGGTATCAACGCAGAGTACXXXXX-3′;

5′-RACE CDS primer (5′-CDS):
(SEQ ID NO: 20)
5′-(T)₂₅VN-3′(N = A, C, G, or T; V = A, G, or C)

Universal primer A mixture (UPM):

(SEQ ID NO: 21)
5′-CTAATACGACTCACTATAGGGCAAGCAGTGGTATCAACGCAGA
GT-3′;

Nested universal primer A (NUP):

(SEQ ID NO: 22)
5′-AAGCAGTGGTATCAACGCAGAGT-3′;
MIgG-CKR:
(SEQ ID NO: 23)
5′-CTAACACTCATTCCTGTTGAAGC-3′;
MIgG-CHR:
(SEQ ID NO: 24)
5′-TCATTTACCCGGAGICCGGGAGAAGCTC-3′.

2. Antibody Variable Region Gene Cloning and Sequencing

RNA extracted from the hybridoma cell strain secreting anti-dengue virus NS1 11E2 monoclonal antibody was subjected to first strand cDNA synthesis using SMARTER™ RACE cDNA amplification kit and SMARTER II A oligonucleotide and 5′-CDS primer in the kit, and the obtained first strand cDNA product is used as PCR amplification template. The light chain gene was amplified with Universal Primer A Mix (UPM), Nested Universal Primer A (NUP) and MIgG-CKR primers, and the heavy chain gene was amplified with Universal Primer A Mix (UPM), Nested Universal Primer A (NUP) and MIgG-CHR primers. A target band of about 0.72 KB was amplified with a light chain primer pair, and a target band of about 1.4 KB was amplified with a heavy chain primer pair. After purification and recovery by agarose gel electrophoresis, the products is subjected to A-Tailing reaction with rTaq DNA polymerase, and then inserted into pMD-18T vector, and transformed into DH5a competent cells; after the cells grew as colonies, 4 clones of each of the heavy and light chain gene clones were selected and sent to Invitrogen Corporation for sequencing.

3. Sequence Analysis of Variable Region Gene of Anti-Dengue Virus NS1 11E2 Antibody

The gene sequence obtained by sequencing was put in the IMGT antibody database for analysis, VNTI111.5 software was used for analysis to determine that the genes amplified by the heavy chain primer pair and the light chain primer pair were correct, where in the gene segment amplified by the light chain, VL gene sequence is 339 bp, and belongs to a VkII gene family, and is preceded by a leader peptide sequence of 60 bp; in the gene fragment amplified by the heavy chain primer pair, VH gene sequence is 354 bp, and belongs to a VH1 gene family, and is preceded by a leader peptide sequence of 57 bp.

4. Construction of Recombinant Antibody Expression Plasmid

pcDNA 3.4 ^{TOP}® vector was a constructed recombinant antibody eukaryotic expression vector, and multiple cloning restriction sites such as HindIII, BamHI, and EcoRI were introduced into the expression vector and named as pcDNA 3.4A expression vector, which was called as 3.4A expression vector for short; according to the sequencing result of the antibody gene in the pMD-18T, the heavy chain and light chain gene specific primers of the anti-dengue virus NS1 11E2 antibody were designed, with the two ends of the primers respectively carrying HindIII and EcoRI restriction sites and protective bases, and the primers were as follows:

DN11E2-HF:
(SEQ ID NO: 25)
5′-CATAAGCTTATGAAATGGAGCTGGGTTATCCTCTTCTTCC-3′;
DN11E2-HR:
(SEQ ID NO: 26)
5′-GACGAATTCTCATTTACCCGGAGTCCGGGAGAAGCTC-3′;
DN11E2-LF:
(SEQ ID NO: 27)
5′-CCCAAGCTTATGGAGACAGACACACTCCTGCTATGG-3′;
DN11E2-LR:
(SEQ ID NO: 28)
5′-CCCGAATTCCTAACACTCATTCCTGTTGAAGCTCTTGAC-3′.

A 0.72 KB of light chain gene fragment and a 1.4 KB of heavy chain gene fragment were amplified by PCR amplification. The heavy chain and light chain gene fragments were subjected to double digestion with HindIII/EcoRI, respectively, and the 3.4A vector was subjected to double digestion with HindIII/EcoRI; after purification and recovery, the heavy chain gene and light chain gene were respectively ligated into the 3.4A expression vector to obtain the recombinant expression plasmids of the heavy chain and light chain, respectively.

5. Screening Stable Cell Strain

5.1 Plasmids were diluted to 400 ng/ml with ultrapure water, CHO cells were adjusted to 1.43×10⁷ cells/ml in a centrifuge tube, 100 μl plasmid was mixed with 700 μl cells, the mixture was subjected to electroporation in an electroporation cup, followed by sampling and counting on days 3, 5 and 7, and collecting samples for detection on day 7.

100 μL/well coating solution was added to dilute the corresponding antigen to a specified concentration, standing overnight at 4° C.; on the next day, the plate was rinsed with a cleaning solution for 2 times, and patted to dry; 120 μL/well blocking solution (20% BSA+80% PBS) was added, standing for 1 h at 37° C., and patted to dry; 100 μL/well diluted cell supernatant was added, standing for 30 min (partial supernatant for 1 h) at 37° C.; the plate was rinsed with a cleaning solution 5 times, and patted to dry; 100 μL/well goat anti-mouse IgG-HRP was added, standing for 30 min at 37° C.; the plate was rinsed with a cleaning solution 5 times, and patted to dry; developing solution A (50 μL/well) and developing solution B (50 μL/well) were added, standing for 10 min; 50 μL/well stop solution was added into the mixture; and OD readings were read at 450 nm (referring to 630 nm) on a microplate reader. A standard curve of standard concentration versus OD value was plotted, and the antibody content in the cell supernatant was calculated.

5.2 Linearization of Recombinant Antibody Expression Plasmid

The reagents buffer 50 μL, DNA 100 μg/tube, and Puv I enzyme 10 μL were prepared, the mixture was supplemented to 500 μL with sterile water, and subjected to enzyme digestion in a 37° C. water bath overnight; the mixture was firstly extracted with an equal volume of phenol/chloroform/isoamyl alcohol (lower layer) of 25:24:1 and then with chloroform (aqueous phase) successively, and precipitated with 0.1 times volume (water phase) of 3 M sodium acetate and 2 times volume of ethanol on ice; the precipitate was rinsed with 70% ethanol to remove the organic solvent, then redissolved with proper amount of sterilized water when the ethanol volatilized completely, and finally subjected to concentration determination.

Stable transfection of the recombinant antibody expression plasmid, and screening a stable cell strain under pressure:

the plasmid was diluted with ultrapure water to 400 ng/ml, CHO cells was adjusted to 1.43×10⁷ cells/ml, 100 μL plasmid was mixed with 700 μL cells, the mixture was subjected to electroporation in an electroporation cup, followed by counting the next day; the mixture was cultured in 25 μmol/L MSX 96-well under pressure for about 25 days.

Cloning wells with cells were observed and marked under a microscope, and the confluence was recorded; the culture supernatant was sampled for detection; cell strains with high antibody concentration and relative concentration were selected, transferred to 24 wells, and transferred to 6 wells around 3 days; after 3 days, preservation and batch culture were performed, the cell density was adjusted to 0.5×10⁶ cells/ml, then batch culture was performed in 2.2 ml culture solution with a cell density of 0.3×10⁶ cells/ml, and 2 ml culture solution was taken for preservation; after 7 days, 6-well batch culture supernatants were sampled for detection, and cell strains with smaller antibody concentration and cell diameter were selected to be transferred to TPP for preservation and passage.

6. Production of Recombinant Antibodies

6.1 Cell Expansion After the cells were thawed, the cells were firstly cultured in a shake flask with a specification of 125 mL, an inoculation volume of 30 mL, and a culture medium of 100% Dynamis culture medium, and the shake flask were placed in a shaking table with a rotating speed of 120 r/min, a temperature of 37° C. and carbon dioxide of 8%. The cells were inoculated and expanded at an inoculation density of 5×10⁵ cells/ml for culturing 72 h, where the expanding volume was calculated according to production requirements, and the culture medium was 100% Dynamis culture medium. The cell expansion was then performed every 72 h. When the cell quantity met the production requirement, the inoculation density was strictly controlled to be about 5×10⁵ cells/ml for production.

6.2 Shake Flask Production and Purification

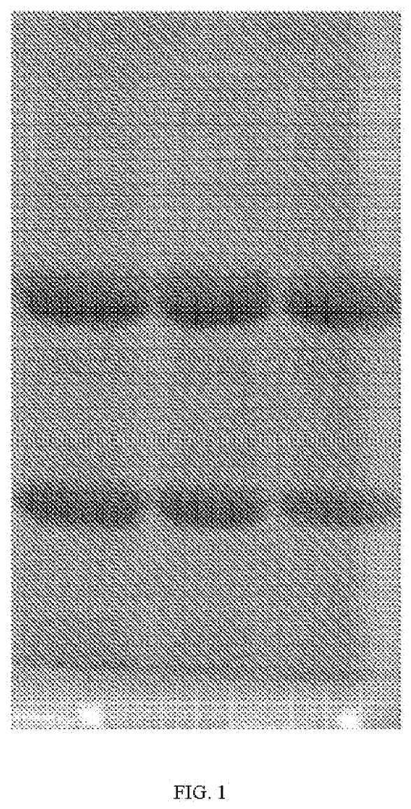

Shake flask parameters: rotational speed 120 r/min, temperature 37° C. and carbon dioxide 8%. Feeding in a flowing manner: daily feeding was started at 72 h in the shake flask, HyClone™ Cell Boost™ Feed 7a was fed daily at an amount of 3% of the initial culture volume, Feed7b was fed daily at an amount of one thousandth of the initial culture volume until day 12 (feeding on day 12). Glucose was supplemented at 3 g/L on day 6. The samples were collected on day 13. Affinity purification was performed on a protein A affinity column. After purification, 500 mg of recombinant antibody was obtained, and 4 μg of purified antibody was subjected to reductive SDS-PAGE, with the electropherogram shown in FIG. 1. Two bands were shown after reductive SDS-PAGE, one for the light chain of about 24 KD (sequence shown in SEQ ID NO: 11) and the other for the heavy chain of about 49 KD (sequence shown in SEQ ID NO: 12).

### Example 2

Although the antibody obtained in Example 1 (having the light chain and heavy chain sequences shown in SEQ ID NO: 11 and SEQ ID NO: 12) had the ability to bind to the NS1 protein, neither affinity nor antibody activity was ideal, and thus the inventors performed mutation analysis and design on the light chain CDR and heavy chain CDR of the antibody.

The complementarity determining region of heavy chain (WT) was analyzed as follows:

CDR-VH1 was G-Y-S(X1)-F-T-D(X2)-Y-W-I-G;
CDR-VH2 was D-M-V(X1)-P-G-D-L(X2)-Y-I-N-Y-Q(X3)-E-
K-F-K-G;
CDR-VH3 was T-N-F-I(X1)-T-V(X2)-G-G-L(X3)-D-Y;

the complementarity determining regions of light chain:

CDR-VL1 was K-S-S-Q(X1)-S-L-L-E(X2)-S-D-G-R(X3)-T-
Y-L-N;
CDR-VL2 was L-V-T(X1)-K-V(X2)-D-S;
CDR-VL3 was W-Q(X1)-G-T-H-F-A(X2)-H-T;

X1, X2 and X3 were all mutation sites.

TABLE 1
Mutation sites associated with antibody activity

| Sites | CDR-VH1 | CDR-VH2 | CDR-VH3 | CDR-VL1 | CDR-VL2 | CDR-VL3 |
|---|---|---|---|---|---|---|
| WT | S | V | V | E | T | A |
| Mutation1 | T | F | L | D | S | P |
| Mutation2 | T | Y | I | E | S | P |
| Mutation3 | T | W | V | S | T | F |
| Mutation4 | V | L | G | W | N | S |
| Mutation5 | F | G | E | Y | P | Y |

The antibody activity was detected after mutation, goat anti-mouse IgG was diluted to 1 μg/mL with 100 μL/well coating solution to carry out microplate coating, standing overnight at 4° C.; on the next day, the plate was rinsed with a cleaning solution for 2 times, and patted to dry; 120 μL/well blocking solution (20% BSA+80% PBS) was added, standing for 1 h at 37° C., and patted to dry; 100 μL/well diluted DN monoclonal antibody was added, standing for 60 min at 37° C.; the liquid in the plate was thrown away, and patted to dry, 120 μL/well 20% mouse negative blood was added for blocking, standing for 1 h at 37° C.; the liquid in the plate was thrown away, and patted to dry, and 100 μL/well (DN-(I+II+III+IV)-NS) antigen (self-produced, expressed in insects) diluted by 10 times, standing for 40 min at 37° C.; the plate was rinsed with a cleaning solution 5 times, and patted to dry; 100 μL/well another DN monoclonal antibody (1:4K) labeled with HRP was added, standing for 30 min at 37° C.; developing solution A (50 μL/well) and developing solution B (50 μL/well) were added, standing for 10 min; 50 μL/well stop solution was added; OD readings were read at 450 nm (referring to 630 nm) on a microplate reader. The partial results were as follows:

TABLE 2
Antibody activity assay data

| Antibody | | | | | | |
|---|---|---|---|---|---|---|
| concentration (ng/ml) | WT | Mutation 1 | Mutation 2 | Mutation 3 | Mutation 4 | Mutation 5 |
| 1000 | 2.316 | 2.403 | 2.075 | 0.847 | 0.514 | — |
| 333.333 | 2.376 | 2.413 | 2.153 | 0.347 | 0.061 | — |
| 111.111 | 2.198 | 2.331 | 1.998 | 0.056 | — | — |
| 37.037 | 1.655 | 1.806 | 1.414 | — | — | — |
| 12.346 | 0.838 | 0.970 | 0.645 | — | — | — |
| 4.115 | 0.448 | 0.453 | 0.234 | — | — | — |
| 1.372 | 0.297 | 0.315 | 0.147 | — | — | — |
| 0 | 0.163 | 0.191 | 0.078 | — | — | — |

“—” represents no activity.

From the above table, it can be seen that mutation had the best activity effect, so mutation 1 was taken as a skeleton sequence to screen mutation sites with better titer (ensuring that the screened antibody activity was similar to that of mutation 1, and the antibody activity was ±10%), with partial results as follows.

TABLE 3
Mutation sites associated with antibody affinity

| | CDR-VH1 | CDR-VH2 | CDR-VH3 | CDR-VL1 | CDR-VL2 | CDR-VL3 |
|---|---|---|---|---|---|---|
| Sites | X2 | X2/X3 | X1/X3 | X1/X3 | X2 | X1 |
| Mutation1 | D | L/Q | I/L | Q/R | V | Q |
| Mutation1-1 | E | L/N | I/L | Q/K | I | Y |
| Mutation 1-2 | D | V/Q | I/V | N/R | L | W |
| Mutation 1-3 | E | V/N | L/I | N/K | V | Q |
| Mutation1-4 | D | I/Q | L/L | Q/R | I | Y |
| Mutation1-5 | E | I/N | L/V | Q/K | L | W |
| Mutation1-6 | D | L/N | I/I | Q/R | V | Q |
| Mutation1-7 | E | L/Q | I/L | Q/K | I | Y |
| Mutation1-8 | D | L/N | I/V | N/R | L | W |
| Mutation1-9 | E | V/Q | L/I | N/K | V | Q |
| Mutation1-10 | D | V/N | L/L | Q/R | I | Y |
| Mutation1-11 | E | I/Q | L/V | Q/K | L | W |
| Mutation1-12 | D | I/N | I/I | Q/R | V | Q |
| Mutation1-13 | E | V/Q | I/V | Q/R | I | Y |
| Mutation1-14 | D | V/N | I/I | Q/K | L | Q |
| Mutation1-15 | E | I/Q | I/I | N/R | V | Q |
| Mutation1-16 | D | I/N | L/L | N/K | I | Y |
| Mutation1-17 | E | V/N | I/L | Q/R | L | W |
| Mutation1-18 | D | I/Q | I/V | Q/K | V | Q |
| Mutation1-19 | E | I/N | I/I | N/R | I | Y |
| Mutation1-20 | D | I/Q | I/L | Q/R | L | W |
| Mutation1-21 | E | I/N | I/V | Q/K | V | Q |
| Mutation1-22 | D | I/N | I/L | Q/R | I | Y |
| Mutation1-23 | E | L/Q | I/V | Q/R | L | W |
| Mutation1-24 | D | L/N | I/I | Q/K | V | Q |
| Mutation1-25 | E | V/Q | L/L | N/R | I | Y |
| Mutation1-26 | D | V/N | L/V | N/K | L | W |
| Mutation1-27 | E | I/Q | L/I | Q/K | V | Q |
| Mutation1-28 | D | I/N | I/I | N/R | I | Y |
| Mutation1-29 | E | L/Q | L/L | N/K | L | W |
| Mutation1-30 | E | L/N | L/V | N/K | I | W |
| Mutation1-31 | E | V/Q | L/I | Q/K | I | Y |
| Mutation1-32 | D | V/N | L/L | N/R | L | W |
| Mutation1-33 | E | I/Q | L/V | N/K | V | Q |
| Mutation1-34 | D | I/N | L/I | Q/R | I | Y |
| Mutation1-35 | E | L/Q | L/V | Q/K | L | W |
| Mutation1-36 | D | L/N | L/I | N/R | V | Q |
| Mutation1-37 | E | V/Q | L/I | N/K | I | Y |
| Mutation1-38 | D | V/N | I/L | Q/R | L | W |
| Mutation1-39 | E | I/Q | I/V | Q/K | V | Q |
| Mutation1-40 | D | V/Q | I/I | N/R | I | Y |
| Mutation1-41 | E | L/Q | L/L | N/K | L | Y |
| Mutation1-42 | D | L/N | L/V | Q/R | V | Q |
| Mutation1-43 | E | V/Q | L/I | Q/K | I | Q |
| Mutation1-44 | D | V/N | I/L | N/R | L | W |
| Mutation1-45 | E | I/Q | I/V | N/K | V | Q |
| Mutation1-46 | E | I/N | I/I | N/R | V | Y |
| Mutation1-47 | E | L/Q | L/L | N/K | L | W |
| Mutation1-48 | D | L/N | L/V | N/R | V | Q |
| Mutation1-49 | E | V/Q | L/I | N/K | I | W |
| Mutation1-50 | D | V/N | I/V | N/K | L | W |
| Mutation1-51 | D | L/Q | I/I | Q/R | V | Q |
| Mutation1-52 | E | V/Q | L/V | Q/R | L | W |

Affinity Analysis

With the AMO sensor, the purified antibody was diluted to 10 ug/ml by PBST, and the DN quality control product recombinant protein (a recombinant antigen produced by the company itself) was subjected to gradient dilution by PBST: 465.1 nmol/ml, 232.6 nmol/ml, 116.3 nmol/ml, 58.1 nmol/ml, 29.1 nmol/ml, 14.5 nmol/ml, 7.27 nmol/ml, and 0 nmol/ml.

Run Process: after equilibration for 60 s in buffer 1 (PBST), antibody was immobilized for 300 s in an antibody solution, incubated for 180 s in buffer 2 (PBST), bound for 420 s in an antigen solution, and dissociated for 1200 s in buffer 2, and sensor regeneration was performed with 10 mM GLY solution at pH 1.69 and buffer 3, and data were output. KID represents the equilibrium dissociation constant, i.e., affinity; Kon represents the binding rate; Koff represents the dissociation rate.

TABLE 4
Affinity Analysis Data

| Sites | KD(M) | Kon (1/Ms) | Koff (1/S) |
|---|---|---|---|
| Mutation 1 | 1.554E−09 | 7.927E+04 | 1.232E−04 |
| Mutation 1-1 | 7.726E−09 | 6.584E+04 | 5.087E−04 |
| Mutation 1-2 | 8.838E−09 | 7.619E+04 | 6.734E−04 |
| Mutation 1-3 | 9.719E−09 | 6.198E+04 | 6.024E−04 |
| Mutation 1-4 | 9.488E−09 | 4.774E+04 | 4.530E−04 |
| Mutation 1-5 | 4.871E−09 | 5.717E+04 | 2.785E−04 |
| Mutation 1-6 | 8.176E−10 | 4.942E+04 | 4.041E−05 |
| Mutation 1-7 | 9.672E−09 | 8.666E+04 | 8.382E−04 |
| Mutation 1-8 | 5.031E−09 | 7.333E+04 | 3.689E−04 |
| Mutation 1-9 | 7.232E−09 | 3.765E+04 | 2.723E−04 |
| Mutation 1-10 | 4.352E−10 | 2.835E+04 | 1.234E−05 |
| Mutation 1-11 | 2.821E−09 | 3.485E+04 | 9.831E−05 |
| Mutation 1-12 | 5.356E−09 | 5.656E+04 | 3.029E−04 |
| Mutation 1-13 | 6.239E−09 | 5.164E+04 | 3.222E−04 |
| Mutation 1-14 | 3.546E−09 | 4.241E+04 | 1.504E−04 |
| Mutation 1-15 | 8.619E−09 | 3.378E+04 | 2.912E−04 |
| Mutation 1-16 | 3.249E−09 | 4.492E+04 | 1.465E−04 |
| Mutation 1-17 | 4.087E−09 | 5.330E+04 | 2.178E−04 |
| Mutation 1-18 | 6.896E−09 | 4.570E+04 | 3.151E−04 |
| Mutation 1-19 | 4.716E−09 | 5.917E+04 | 2.790E−04 |
| Mutation 1-20 | 1.891E−09 | 5.020E+04 | 9.490E−05 |
| Mutation 1-21 | 4.601E−09 | 8.734E+04 | 4.018E−04 |
| Mutation 1-22 | 9.876E−10 | 2.400E+04 | 2.370E−05 |
| Mutation 1-23 | 2.779E−09 | 4.824E+04 | 1.341E−04 |
| Mutation 1-24 | 3.540E−10 | 2.165E+04 | 7.665E−06 |
| Mutation 1-25 | 6.039E−09 | 4.514E+04 | 2.726E−04 |
| Mutation 1-26 | 7.467E−09 | 5.287E+04 | 3.948E−04 |
| Mutation 1-27 | 2.305E−09 | 6.827E+04 | 1 574E−04 |
| Mutation 1-28 | 6.592E−09 | 5.118E+04 | 3.374E−04 |
| Mutation 1-29 | 7.943E−09 | 2.325E+04 | 1.847E−04 |
| Mutation 1-30 | 5.021E−09 | 2.269E+04 | 1.139E−04 |
| Mutation 1-31 | 8.220E−09 | 5.848E+04 | 4.807E−04 |
| Mutation 1-32 | 8.655E−09 | 3.594E+04 | 3.111E−04 |
| Mutation 1-33 | 9.614E−09 | 6.228E+04 | 5.988E−04 |
| Mutation 1-34 | 4.940E−10 | 5.470E+04 | 2.702E−05 |
| Mutation 1-35 | 4.004E−09 | 4.753E+04 | 1.903E−04 |
| Mutation 1-36 | 6.466E−09 | 6.416E+04 | 4.149E−04 |
| Mutation 1-37 | 8.896E−09 | 8.636E+04 | 7.683E−04 |
| Mutation 1-38 | 3.472E−09 | 7.387E+04 | 2.565E−04 |
| Mutation 1-39 | 2.324E−10 | 5.196E+04 | 1.208E−05 |
| Mutation 1-40 | 4.051E−09 | 4.230E+04 | 1.714E−04 |
| Mutation 1-41 | 3.875E−10 | 3.022E+04 | 1.171E−05 |
| Mutation 1-42 | 1.703E−09 | 8.932E+04 | 1.521E−04 |
| Mutation 1-43 | 5.851E−09 | 2.254E+04 | 1.319E−04 |
| Mutation 1-44 | 4.263E−09 | 5.708E+04 | 2.433E−04 |
| Mutation 1-45 | 6.592E−09 | 6.527E+04 | 4.302E−04 |
| Mutation 1-46 | 8.002E−09 | 5.991E+04 | 4.794E−04 |
| Mutation 1-47 | 6.121E−10 | 2.749E+04 | 1.683E−05 |
| Mutation 1-48 | 5.609E−09 | 4.111E+04 | 2.306E−04 |
| Mutation 1-49 | 7.953E−09 | 6.449E+04 | 5.129E−04 |
| Mutation 1-50 | 6.051E−09 | 7.696E+04 | 4.657E−04 |
| Mutation 1-51 | 2.687E−09 | 4.289E+04 | 1.152E−04 |
| Mutation 1-52 | 1.554E−09 | 7.927E+04 | 1.232E−04 |

The above experiments were repeated using WT as the backbone sequence and affinity verification of the mutation sites was performed, with partial results as follows.

TABLE 5
Mutations with WT as backbone

| | CDR-VH1 | CDR-VH2 | CDR-VH3 | CDR-VL1 | CDR-VL2 | CDR-VL3 |
|---|---|---|---|---|---|---|
| Sites | X2 | X2/X3 | X1/X3 | X1/X3 | X2 | X1 |
| WT | D | L/Q | I/L | Q/R | V | Q |
| WT 1-1 | E | L/N | I/V | Q/K | I | Y |
| WT 1-4 | D | I/Q | L/V | Q/R | I | Y |
| WT 1-7 | E | L/Q | I/V | Q/K | I | Y |
| WT 1-20 | D | I/Q | I/L | Q/R | L | W |
| WT 1-30 | E | L/N | L/V | N/K | I | W |

TABLE 6
Affinity Analysis Data

| Sites | KD(M) | Kon (1/Ms) | Koff (1/S) |
|---|---|---|---|
| WT | 5.884E−08 | 6.771E+03 | 3.984E−04 |
| WT 1-1 | 1.282E−08 | 4.568E+03 | 5.856E−05 |
| WT 1-4 | 2.798E−08 | 5.079E+03 | 1.421E−04 |
| WT 1-7 | 6.550E−08 | 3.528E+03 | 2.311E−04 |
| WT 1-20 | 4.587E−08 | 3.121E+03 | 1.432E−04 |
| WT 1-30 | 2.041E−08 | 7.284E+03 | 1.487E−04 |

From the analysis in Table 5 and Table 6, under the premise of ensuring the antibody activity, the affinities of antibodies based on mutation 1 as the framework were overall higher than that of WT.

### Example 3

The foregoing antibody was placed in 4° C. (refrigerator), −80° C. (refrigerator) and 37° C. (incubator) for 21 days. The samples were taken to observe the state of the antibody on day 7, 14 and 21, and the activity of the samples was detected on day 21. The results showed that there was no obvious change in the state of the antibody after 21 days in the three assessment conditions, the activity also did not decrease with the increase of the assessment temperature, indicating that the self-produced antibody was stable; and the following table shows the OD results of enzyme-linked immunoassay for 21-day assessment.

TABLE 7
Antibody stability assessment

| Sample concentration (ug/ml) | 3 | 0.75 | 0 |
|---|---|---|---|
| 4° C., 21-day samples | 2.432 | 2.043 | 0.075 |
| −80° C., 21-day samples | 2.546 | 2.054 | 0.057 |
| 37° C., 21-day samples | 2.499 | 2.033 | 0.089 |

Finally, it should be noted that the foregoing examples are merely intended for describing the technical solutions of the present disclosure, but not for limiting the present disclosure. Although the present disclosure is described in detail with reference to the foregoing examples, persons of ordinary skill in the art should understand that they may still make modifications to the technical solutions described in the foregoing examples or make equivalent replacements to some or all technical features thereof, without departing from the scope of the technical solutions of the examples of the present disclosure.

### INDUSTRIAL APPLICABILITY

The present disclosure provides an isolated binding protein including an antigen binding domain binding to the NS1 protein, the binding protein with higher sensitivity and specificity can specifically identify and bind to the NS1 protein, thereby enabling detection of dengue virus. Moreover, there is no need to use mouse peritoneal cavity to induce hybridoma cells to produce the binding protein, which has the advantages of being less difficult in the production, and more stable in the antibody function.

What is claimed is:

1. An isolated binding protein comprising an antigen binding domain, wherein the antigen binding domain comprises at least one complementarity determining region selected from the following amino acid sequences,
or has at least 80% sequence identity with the complementarity determining region of the following amino acid sequences and has an affinity of K_{D}≤6.55×10⁻⁸ mol/L to a NS1 protein;
the complementarity determining region CDR-VH1 is G-Y-X1-F-T-X2-Y-W-1-G, wherein,
X1 is S or T, and X2 is D or E;
the complementarity determining region CDR-VH2 is D-M-X1-P-G-D-X2-Y-1-N-Y-X3-E-K-F-K-G, wherein,
X1 is F or V, X2 is V, L or I, and X3 is Q or N;
the complementarity determining region CDR-VH3 is T-N-F-X1-T-X2-G-G-X3-D-Y, wherein,
X1 is I or L, X2 is L or V, and X31 is V, L or I;
the complementarity determining region CDR-VL1 is K-S-S-X1-S-L-L-X2-S-D-G-X3-T-Y-L-N, wherein,
X1 is Q or N, X2 is E or D, and X3 is R or K;
the complementarity determining region CDR-VL2 is L-V-X1-K-X2-D-S, wherein,
X1 is S or T, and X2 is V, I or L;
the complementarity determining region CDR-VL3 is W-X1-G-T-H-F-X2-H-T, wherein,
X1 is Q, Y or W, and X2 is A or P.

2. The binding protein according to claim 1, wherein the amino acids at the corresponding sites of the complementarity determining regions are listed below,

| | CDR-VH1 | CDR-VH2 | CDR-VH3 | CDR-VL1 | CDR-VL2 | CDR-VL3 |
|---|---|---|---|---|---|---|
| Sites | X2 | X2/X3 | X1/X3 | X1/X3 | X2 | X1 |
| Mutation 1 | D | L/Q | I/L | Q/R | V | Q |
| Mutation 1-1 | E | L/N | I/L | Q/K | I | Y |
| Mutation 1-2 | D | V/Q | I/V | N/R | L | W |
| Mutation 1-3 | E | V/N | L/I | N/K | V | Q |
| Mutation 1-4 | D | I/Q | L/L | Q/R | I | Y |
| Mutation 1-5 | E | I/N | L/V | Q/K | L | W |
| Mutation 1-6 | D | L/N | I/I | Q/R | V | Q |
| Mutation 1-7 | E | L/Q | I/L | Q/K | I | Y |
| Mutation 1-8 | D | L/N | I/V | N/R | L | W |
| Mutation 1-9 | E | V/Q | L/I | N/K | V | Q |
| Mutation 1-10 | D | V/N | L/L | Q/R | I | Y |
| Mutation 1-11 | E | I/Q | L/V | Q/K | L | W |
| Mutation 1-12 | D | I/N | I/I | Q/R | V | Q |
| Mutation 1-13 | E | V/Q | I/V | Q/R | I | Y |
| Mutation 1-14 | D | V/N | I/I | Q/K | L | Q |
| Mutation 1-15 | E | I/Q | I/I | N/R | V | Q |
| Mutation 1-16 | D | I/N | L/L | N/K | I | Y |
| Mutation 1-17 | E | V/N | I/L | Q/R | L | W |
| Mutation 1-18 | D | I/Q | I/Y | Q/K | V | Q |
| Mutation 1-19 | E | I/N | I/I | N/R | I | Y |
| Mutation 1-20 | D | I/Q | I/L | Q/R | L | W |
| Mutation 1-21 | E | I/N | I/Y | Q/K | V | Q |
| Mutation 1-22 | D | I/N | I/L | Q/R | I | Y |
| Mutation 1-23 | E | L/Q | I/V | Q/R | L | W |
| Mutation 1-24 | D | L/N | I/I | Q/K | V | Q |
| Mutation 1-25 | E | V/Q | L/L | N/R | I | Y |
| Mutation 1-26 | D | V/N | L/V | N/K | L | W |
| Mutation 1-27 | E | I/Q | L/I | Q/K | V | Q |
| Mutation 1-28 | D | I/N | I/I | N/R | I | Y |
| Mutation 1-29 | E | L/Q | L/L | N/K | L | W |
| Mutation 1-30 | E | L/N | L/V | N/K | I | W |
| Mutation 1-31 | E | V/Q | L/I | Q/K | I | Y |
| Mutation 1-32 | D | V/N | L/L | N/R | L | W |
| Mutation 1-33 | E | I/Q | L/V | N/K | V | Q |
| Mutation 1-34 | D | I/N | L/I | Q/R | I | Y |
| Mutation 1-35 | E | L/Q | L/V | Q/K | L | W |
| Mutation 1-36 | D | L/N | L/I | N/R | V | Q |
| Mutation 1-37 | E | V/Q | L/I | N/K | I | Y |
| Mutation 1-38 | D | V/N | I/L | Q/R | L | W |
| Mutation 1-39 | E | I/Q | I/V | Q/K | V | Q |
| Mutation 1-40 | D | V/Q | I/I | N/R | I | Y |
| Mutation 1-41 | E | L/Q | L/L | N/K | L | Y |
| Mutation 1-42 | D | L/N | L/V | Q/R | V | Q |
| Mutation 1-43 | E | V/Q | L/I | Q/K | I | Q |
| Mutation 1-44 | D | V/N | I/L | N/R | L | W |
| Mutation 1-45 | E | I/Q | I/V | N/K | V | Q |
| Mutation 1-46 | E | I/N | I/I | N/R | V | Y |
| Mutation 1-47 | E | L/Q | L/L | N/K | L | W |
| Mutation 1-48 | D | L/N | L/V | N/R | V | Q |
| Mutation 1-49 | E | V/Q | L/I | N/K | I | W |
| Mutation 1-50 | D | V/N | I/V | N/K | L | W |
| Mutation 1-51 | D | L/Q | I/I | Q/R | V | Q |
| Mutation 1-52 | E | V/Q | L/V | Q/R | L | W |

3. The binding protein according to claim 1, wherein the binding protein comprises at least 3 CDRs; or the binding protein comprises at least 6 CDRs.

4. The binding protein according to claim 1, wherein the binding protein is one of a nanobody, F(ab′)2, Fab′, Fab, Fv, scFv, a bispecific antibody, and an antibody minimum recognition unit.

5. The binding protein according to claim 1, wherein the binding protein further comprises an antibody constant region sequence.

6. An isolated nucleic acid encoding the binding protein according to claim 1.

7. A vector comprising the nucleic acid according to claim 6.

8. A host cell comprising the nucleic acid according to claim 6.

9. A method for producing the binding protein according to claim 1, wherein, the method comprises the following steps:
culturing a host cell comprising an nucleic acid encoding the binding protein according to claim 1 in a culture medium, and recovering the produced binding protein from the culture medium or from the cultured host cell.

10. (canceled)

11. A kit comprising one or more of the binding protein according to claim 1.

12. (canceled)

13. A method for detecting the NS1 protein comprising:
A) contacting a sample from a subject with the binding protein according to claim 1 for performing a binding reaction in a condition sufficient for a binding reaction; and
B) detecting an immune complex generated by the binding reaction,
wherein the presence of the immune complex is indicative of the presence of the NS1 protein.

14. The method according to claim 13, wherein the method is based on a fluorescence immunoassay, a chemiluminescence immunoassay, a colloidal gold immunoassay, a radioimmunoassay and/or an enzyme-linked immunoassay.

15. The method according to claim 13, wherein the sample is selected from at least one of whole blood, peripheral blood, serum, or plasma.

16. The method according to claim 13, wherein the subject is a mammal, preferably a primate, more preferably a human.

17. The binding protein according to claim 1, wherein in the complementarity determining region CDR-VH1, X1 is T
in the complementarity determining region CDR-VH2, X1 is F;
in the complementarity determining region CDR-VH3, X2 is L;
in the complementarity determining region CDR-VL1, X2 is D;
in the complementarity determining region CDR-VL2, X1 is S;
in the complementarity determining region CDR-VL3, X2 is P;
preferably, in the complementarity determining region CDR-VH1 X2 is D;
preferably, in the complementarity determining region CDR-VH1, X2 is E;
preferably, in the complementarity determining region CDR-VH2, X2 is V, and X3 is Q;
preferably, in the complementarity determining region CDR-VH2, X2 is V, and X3 is N;
preferably, in the complementarity determining region CDR-VH2, X2 is L, and X3 is Q;
preferably, in the complementarity determining region CDR-VH2, X2 is L, and X3 is N;
preferably, in the complementarity determining region CDR-VH2, X2 is I, and X3 is Q;
preferably, in the complementarity determining region CDR-VH2, X2 is I, and X3 is N;
preferably, in the complementarity determining region CDR-VH3, X1 is I, and X3 is V;
preferably, in the complementarity determining region CDR-VH3, X1 is I, and X3 is L;
preferably, in the complementarity determining region CDR-VH3, X1 is I, and X3 is I;
preferably, in the complementarity determining region CDR-VH3, X1 is L, and X3 is V;
preferably, in the complementarity determining region CDR-VH3, X1 is L, and X3 is L;
preferably, in the complementarity determining region CDR-VH3, X1 is L, and X3 is I;
preferably, in the complementarity determining region CDR-VL1, X1 is Q, and X2 is R;
preferably, in the complementarity determining region CDR-VL1, X1 is Q, and X2 is K;
preferably, in the complementarity determining region CDR-VL1, X1 is N, and X2 is R;
preferably, in the complementarity determining region CDR-VL1, X1 is N, and X2 is K;
preferably, in the complementarity determining region CDR-VL2, X2 is V;
preferably, in the complementarity determining region CDR-VL2, X2 is I;
preferably, in the complementarity determining region CDR-VL2, X2 is L;
preferably, in the complementarity determining region CDR-VL3, X1 is Q;
preferably, in the complementarity determining region CDR-VL3, X1 is Y;
preferably, in the complementarity determining region CDR-VL3, X1 is W.

18. The binding protein according to claim 5, wherein in the antibody constant region sequence is selected from the sequence of any one of the constant regions of IgG1, IgG2, IgG3, IgG4, IgA, IgM, IgE, and IgD.

19. The binding protein according to claim 5, wherein a species source of the antibody constant region is cow, horse, dairy cow, pig, sheep, goat, rat, mouse, dog, cat, rabbit, camel, donkey, deer, mink, chicken, duck, goose, turkey, gamecock, or human.

20. The binding protein according to claim 5, wherein the antibody constant region is derived from a mouse:
a light chain constant region sequence is shown in SEQ ID NO: 9;
a heavy chain constant region sequence is shown in SEQ ID NO: 10.

21. The binding protein according to claim 1, wherein the binding protein is labeled with an indicator for displaying signal intensity.

22. The binding protein according to claim 4, wherein the binding protein comprises the light chain framework regions FR-L1, FR-L2, FR-L3, and FR-L4 with sequences shown in SEQ ID NOs: 1-4 successively, and/or the heavy chain framework regions FR-H1, FR-H2, FR-H3, and FR-H4 with sequences shown in SEQ ID NOs: 5-8 successively.


SEQUENCE LISTING (28 sequences)

SEQ ID NO: 1 - protein, 23 aa, Mus musculus
DVVMTQTPLTLSVTIGQPASISC

SEQ ID NO: 2 - protein, 15 aa, Mus musculus
WLLQRPGQSPKRLIY

SEQ ID NO: 3 - protein, 32 aa, Mus musculus
GVPDRFTGSGSVTDFTLKISRVESEDLGVYYC

SEQ ID NO: 4 - protein, 9 aa, Mus musculus
FGAGTKLEV

SEQ ID NO: 5 - protein, 25 aa, Mus musculus
QVQLQQSGAELVRPGTSVKISCKAS

SEQ ID NO: 6 - protein, 14 aa, Mus musculus
WVKQRPGHGLEWIG

SEQ ID NO: 7 - protein, 30 aa, Mus musculus
KATLTADTSSNTAYMQLSSLTSEDSAVYFC

SEQ ID NO: 8 - protein, 11 aa, Mus musculus
WGRGTSLTVSS

SEQ ID NO: 9 - protein, 108 aa, Mus musculus
KRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 10 - protein, 330 aa, Mus musculus
AKTTPPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 11 - protein, 219 aa, Mus musculus
DVVMTQTPLTLSVTIGQPASISCKSSNSLLDSDGKTYLNWLLQRPGQSPKRLIYLVTKIDSGVPDRFTGSGSVTDFTLKISRVESEDLGVYYCWWGTHFPHTFGAGTKLEVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 12 - protein, 448 aa, Mus musculus
QVQLQQSGAELVRPGTSVKISCKASGYSFTDYWIGWVKQRPGHGLEWIGDMVPGDLYINYQEKFKGKATLTADTSSNTAYMQLSSLTSEDSAVYFCTNFITVGGLDYWGRGTSLTVSSAKTTPPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 13 - protein, 10 aa, Artificial Sequence
  mutated CDR-VH1
  MISC_FEATURE (3)..(3): Xaa is S or T
  MISC_FEATURE (6)..(6): Xaa is D or E
GYXFTXYWIG

SEQ ID NO: 14 - protein, 17 aa, Artificial Sequence
  mutated CDR-VH2
  MISC_FEATURE (3)..(3): Xaa is F or V
  MISC_FEATURE (7)..(7): Xaa is V, L or I
  MISC_FEATURE (12)..(12): Xaa is Q or N
DMXPGDXYINYXEKFKG

SEQ ID NO: 15 - protein, 11 aa, Artificial Sequence
  mutated CDR-VH3
  MISC_FEATURE (4)..(4): Xaa is I or L
  MISC_FEATURE (6)..(6): Xaa is L or V
  MISC_FEATURE (9)..(9): Xaa is V, L or I
TNFXTXGGXDY

SEQ ID NO: 16 - protein, 16 aa, Artificial Sequence
  mutated CDR-VL1
  MISC_FEATURE (4)..(4): Xaa is Q or N
  MISC_FEATURE (8)..(8): Xaa is E or D
  MISC_FEATURE (12)..(12): Xaa is R or K
KSSXSLLXSDGXTYLN

SEQ ID NO: 17 - protein, 7 aa, Artificial Sequence
  mutated CDR-VL2
  MISC_FEATURE (3)..(3): Xaa is S or T
  MISC_FEATURE (5)..(5): Xaa is V, I or L
LVXKXDS

SEQ ID NO: 18 - protein, 9 aa, Artificial Sequence
  mutated CDR-VL3
  MISC_FEATURE (2)..(2): Xaa is Q, Y or W,
  MISC_FEATURE (7)..(7): Xaa is  A or P
WXGTHFXHT

SEQ ID NO: 19 - DNA, 25 bp, Artificial Sequence
  SMARTER II A oligonucleotide from a commerical kit
  misc_feature (25)..(25): XXXXX at the 3′-end
aagcagtggtatcaacgcagagtac

SEQ ID NO: 20 - DNA, 25 bp, Artificial Sequence
  5′-RACE CDS primer
  misc_feature (25)..(25): n is a, c, g, or t
  misc_feature (26)..(26): na, c, g, or t
  misc_feature (27)..(27): va, c or g
tttttttttttttttttttttttvn

SEQ ID NO: 21 - DNA, 45 bp, Artificial Sequence
  Universal primer A mixture
ctaatacgactcactatagggcaagcagtggtatcaacgcagagt

SEQ ID NO: 22 - DNA, 23 bp, Artificial Sequence
  Nested universal primer A
aagcagtggtatcaacgcagagt

SEQ ID NO: 23 - DNA, 23 bp, Artificial Sequence
  MIgG-CKR
ctaacactcattcctgttgaagc

SEQ ID NO: 24 - DNA, 28 bp, Artificial Sequence
  MIgG-CHR
tcatttacccggagtccgggagaagctc

SEQ ID NO: 25 - DNA, 40 bp, Artificial Sequence
  DN11E2-HF
cataagcttatgaaatggagctgggttatcctcttcttcc

SEQ ID NO: 26 - DNA, 37 bp, Artificial Sequence
  DN11E2-HR
gacgaattctcatttacccggagtccgggagaagctc

SEQ ID NO: 27 - DNA, 36 bp, Artificial Sequence
  DN11E2-LF
cccaagcttatggagacagacacactcctgctatgg

SEQ ID NO: 28 - DNA, 39 bp, Artificial Sequence
  DN11E2-LR
cccgaattcctaacactcattcctgttgaagctcttgac